(19) United States
(12) Patent Application Publication
(10) Pub. No.: US 2026/0193174 A1
(43) Pub. Date: Jul. 9, 2026

(54) METHODS OF MAKING FORMAMIDINIUM IODIDE AND USES THEREOF

(71) Applicant: Minh Tu Nguyen, Morrisville, NC (US)

(72) Inventor: Minh Tu Nguyen, Morrisville, NC (US)

(73) Assignee: PY Materials, LLC, Morrisville, NC (US)

(21) Appl. No.: 19/013,915

(22) Filed: Jan. 8, 2025

(51) Int. Cl. C07C 249/00; H10K 85/50

(52) U.S. Cl. CPC C07C 249/00; H10K 85/50

(57) ABSTRACT

The present disclosure relates to a method for making formamidinium iodide compound. The method comprises mixing a first precursor, which contains formamidinium ions, with a first liquid (step (a)); mixing a second precursor, which contains iodide ions, and a second liquid (step (b)); preparing a reaction mixture (step (c)) by mixing the resulting mixture in step (a) with the resulting mixture in step (b); introducing a third liquid (step (d)) to the reaction mixture in step (c); mixing the reaction mixture (step (e)); collecting the solution which contains formamidinium iodide product (step (f)); and isolating formamidinium iodide product by removing solvents in the product solution (step (g)).

### BACKGROUND

Perovskite solar cell (PSC) has emerged in recent years as the most viable low-cost future solar technology owing to their potential high efficiency, affordability, and ease of manufacturing. Their performance has quickly improved, in some cases rivaling or exceeding that of conventional silicon-based solar cells. These cells hold significant promise for lightweight, flexible, and scalable solar energy applications. Furthermore, perovskite materials are gaining significant attention for their potential in other optoelectronic devices due to their unique optical and electronic properties. These materials are particularly promising for applications in light-emitting diodes (LEDs), lasers, photodetectors, and displays. Perovskites exhibit excellent light absorption, tunable bandgaps, and high charge-carrier mobility, making them ideal for efficient light emission and detection. Their versatility allows for easy integration into various device structures, enabling the development of flexible, high-performance optoelectronic components. Formamidinium-containing perovskites, such as formamidinium lead iodide (FAPbI3), have been commonly used in the fabrication of high-performance solar cells and other optoelectronic devices. To produce formamidinium-containing perovskites, formamidinium iodide (FAI) serves as a key precursor both in solution-processed and solvent-free methods.

### SUMMARY

According to some embodiments, a method for making formamidinium iodide compound comprises mixing a first precursor, which contains formamidinium ions, with a first liquid (step (a)). The method further comprises mixing a second precursor, which contains iodide ions, and a second liquid (step (b)). The method further comprises preparing a reaction mixture (step (c)) by mixing the resulting mixture in step (a) with the resulting mixture in step (b). Further still, the method comprises introducing a third liquid (step (d)) to the reaction mixture in step (c). Further still, the method comprises mixing the reaction mixture (step (e)). Further still, the method comprises collecting the solution which contains formamidinium iodide product. Finally, the method comprises isolating formamidinium iodide product by removing solvents in the product solution.

According to some embodiments, the counterions of formamidinium ions in the first precursor comprises at least one of formate, acetate, propanoate, benzoate, oxalate, lactate, citrate, sulfate, phosphate, nitrate, chloride, or bromide. According to some embodiments, the counterions of iodide ions in the second precursor comprises at least one of Li⁺, Na⁺, K⁺, Rb⁺, Cs⁺, H₃O⁺, NH₄⁺, CH₃NH₃⁺, (CH₃)₄N⁺, Ca²⁺, Mg²⁺, Fe²⁺, Mn²⁺, Zn²⁺, or Al³⁺.

According to some embodiments, the first liquid is selected from the group consisting of water, methanol, ethanol, propanol, butanol, formic acid, acetic acid, acetone, 2-butanone, acetonitrile, acrylonitrile, N-methyl-2-pyrrolidone, dimethylformamide, dimethyl sulfoxide, and combinations thereof. According to some embodiments, the second liquid is the same as the first liquid. According to some embodiments, the second liquid is selected from the group consisting of water, methanol, ethanol, propanol, butanol, formic acid, acetic acid, acetone, 2-butanone, acetonitrile, acrylonitrile, N-methyl-2-pyrrolidone, dimethylformamide, dimethyl sulfoxide, and combinations thereof.

According to some embodiments, the third liquid is selected from the group consisting of water, methanol, ethanol, propanol, butanol, formic acid, acetic acid, acetone, 2-butanone, acetonitrile, acrylonitrile, N-methyl-2-pyrrolidone, dimethylformamide, dimethyl sulfoxide, ethylene glycol, 2-methoxyethanol, 2-ethoxyethanol, 2-propoxyethanol, 2-butoxyethanol, propylene glycol, 1-methoxy-2-propanol, 1-ethoxy-2-propanol, methyl lactate, ethyl lactate, propylene carbonate, 4-butyrolactone, and combinations thereof.

According to some embodiments, the third liquid is the same as the second liquid, which is the same as the first liquid, wherein the first liquid is selected from the group consisting of water, methanol, ethanol, propanol, butanol, formic acid, acetic acid, acetone, 2-butanone, acetonitrile, acrylonitrile, N-methyl-2-pyrrolidone, dimethylformamide, dimethyl sulfoxide, ethylene glycol, 2-methoxyethanol, 2-ethoxyethanol, 2-propoxyethanol, 2-butoxyethanol, propylene glycol, 1-methoxy-2-propanol, 1-ethoxy-2-propanol, methyl lactate, ethyl lactate, propylene carbonate, 4-butyrolactone, and combinations thereof.

According to some embodiments, the first precursor contains cationic impurities comprising at least one of Li⁺, Na⁺, K⁺, Rb⁺, Cs⁺, H₃O⁺, NH₄⁺, CH₃NH₃⁺, (CH₃)₄N⁺, Be²⁺, Ca²⁺, Mg²⁺, Sr²⁺, Ba²⁺, Fe²⁺, Ni²⁺, Mn²⁺, Co²⁺, Zn²⁺, Al³⁺, or Bi³⁺. According to some embodiments, the second precursor contains anionic impurities comprising at least one of formate, acetate, propanoate, butanoate, pentanoate, hexanoate, benzoate, oxalate, malonate, lactate, citrate, hydrogen citrate, dihydrogen citrate, sulfate, hydrogen sulfate, phosphate, hydrogen phosphate, dihydrogen phosphate, nitrate, carbonate, bicarbonate, hydroxide, chloride, or bromide.

According to some embodiments, the temperature of the reaction mixture is adjusted to a temperature greater than or equal to 0° C. and less than or equal to 180° C. According to some embodiments, step (a) and step (b) are omitted and the reaction mixture in step (c) is prepared by mixing the first precursor, the second precursor, the first liquid, and the second liquid all together.

According to some embodiments, the third liquid is introduced in step (d) through a continuous process; wherein the mixture in step (c) and the third liquid are continuously fed into a reactor; wherein the temperature of the reactor is kept at a temperature greater than or equal to 0° C. and less than or equal to 180° C.; and wherein continuously a product mixture comprising formamidinium iodide is removed from the reactor. According to some embodiments, step (c) is omitted and the third liquid is introduced through a continuous process; wherein the mixture in step (a), the mixture in step (b), and the third liquid are continuously fed into a reactor; wherein the temperature of the reactor is kept at a temperature greater than or equal to 0° C. and less than or equal to 180° C.; and wherein continuously a product mixture comprising formamidinium iodide is removed from the reactor. According to some embodiments, a pressure is applied to the reaction mixture, wherein the applied pressure to the reaction mixture is adjusted to a value greater than or equal to 1 bar and less than or equal to 220 bar.

According to some embodiments, the method may further include, after the isolating of formamidinium iodide product, forming a perovskite film, wherein forming the perovskite film utilizes the isolated formamidinium iodide product as a precursor. According to some embodiments, the perovskite film is composed of a perovskite of formula: APbX3 wherein, A is a monovalent cation consisting of at least 70% formamidinium cation by mole fraction; Pb is lead(ii) cation; X is a monovalent halide anion consisting of at least 70% iodide anion by mole fraction. According to some embodiments, an optoelectronic and/or photoelectrochemical device is fabricated, wherein the optoelectronic and/or photoelectrochemical device comprises the perovskite film manufactured from the isolated formamidinium iodide precursor.

### BRIEF DESCRIPTION OF THE DRAWINGS

FIG. 1 is a flowchart for an example method of making formamidinium iodide (FAI).

### DETAILED DESCRIPTION OF PREFERRED EMBODIMENTS

The methods described in the present disclosure for making formamidinium iodide compound may be used to manufacture one or more aspects of perovskite solar cells. Other potential applications include, but are not limited to, light-emitting diodes (LEDs), lasers, photodetectors, phototransistors, gas sensors, batteries, field-effect transistors (FETs), photocatalysis, water splitting, and/or thermoelectric devices. Formamidinium iodide (FAI) has other names include, but are not limited to, iminomethylamine hydriodide, methanimidamide iodide, or formamidine hydroiodide. Formamidinium iodide may be identified by Chemical Abstracts Service (CAS) Registry Number: 879643-71-7.

In some embodiments, the present disclosure provides methods of making formamidinium iodide compound based on solution processing, wherein a mixture containing two or more components is a solution. In further embodiments, the present disclosure provides methods of making formamidinium iodide compound using a suspension-based approach, wherein a prepared mixture contains components in both solid state and liquid state. In further embodiments, the present disclosure provides methods of making formamidinium iodide compound involves heterogeneous liquid mixtures, wherein a prepared mixture is a combination of liquids in which the components do not blend uniformly, leading to the formation of distinct layers.

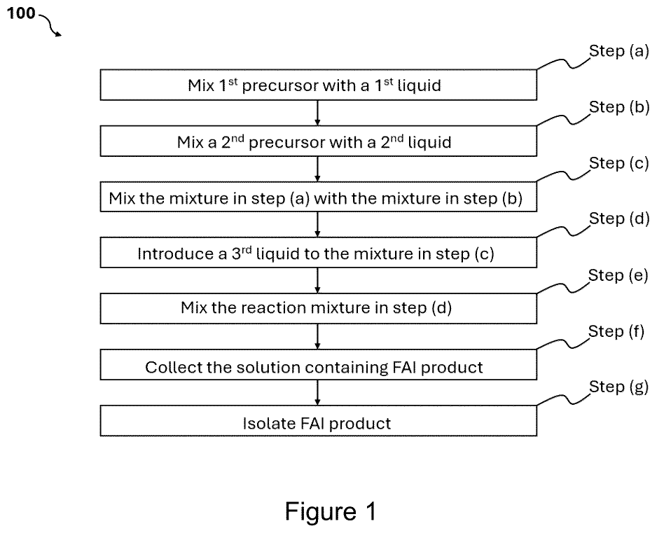

FIG. 1 illustrates an exemplary method 100 for making formamidinium iodide compound. Step (a) comprises mixing a first precursor, which contains formamidinium ions, with a first liquid. The first precursor is an organic salt of formamidinium cation [(H₂N═CH(NH₂)⁺] and a counterion, which comprises at least one of formate, acetate, propanoate, benzoate, oxalate, lactate, citrate, sulfate, phosphate, nitrate, chloride, or bromide. The first liquid is selected from the group consisting of water, methanol, ethanol, propanol, butanol, formic acid, acetic acid, acetone, 2-butanone, acetonitrile, acrylonitrile, N-methyl-2-pyrrolidone, dimethylformamide, dimethyl sulfoxide, and combinations thereof. In one embodiment, the resulting mixture in step (a) is a solution. In other embodiments, the resulting mixture in step (a) is a suspension. In some embodiments, mixing in step (a) is carried out using at least one of mechanical, ultrasonic, vibration, pneumatic, ball-milling, and high-shear mixing methods. In other embodiments, the first precursor contains cationic impurities comprising at least one of Li⁺, Na⁺, K⁺, Rb⁺, Cs⁺, H₃O⁺, NH₄⁺, CH₃NH₃⁺, (CH₃)₄N⁺, Be²⁺, Ca²⁺, Mg²⁺, Sr²⁺, Ba²⁺, Fe²⁺, Ni²⁺, Mn²⁺, Co²⁺, Zn²⁺, Al³⁺, or Bi³⁺. Step (b) comprises mixing a second precursor, which contains iodide ions, with a second liquid. The second precursor is a salt composed of iodide (I⁻) anions and counterions, which comprise at least one of Li⁺, Na⁺, K⁺, Rb⁺, Cs⁺, H₃O⁺, NH₄⁺, CH₃NH₃⁺, (CH₃)₄N⁺, Ca²⁺, Mg²⁺, Fe²⁺, Mn²⁺, Zn²⁺,or Al³⁺. The second liquid is selected from the group consisting of water, methanol, ethanol, propanol, butanol, formic acid, acetic acid, acetone, 2-butanone, acetonitrile, acrylonitrile, N-methyl-2-pyrrolidone, dimethylformamide, dimethyl sulfoxide, and combinations thereof. In some embodiments, the second liquid could be the same as the first liquid in step (a). In one embodiment, the resulting mixture in step (b) is a solution. In other embodiments, the resulting mixture in step (b) is a suspension. In some embodiments, mixing in step (b) is carried out using at least one of mechanical, ultrasonic, vibration, pneumatic, ball-milling, and high-shear mixing methods. In other embodiments, the second precursor contains anionic impurities comprising at least one of formate, acetate, propanoate, butanoate, pentanoate, hexanoate, benzoate, oxalate, malonate, lactate, citrate, hydrogen citrate, dihydrogen citrate, sulfate, hydrogen sulfate, phosphate, hydrogen phosphate, dihydrogen phosphate, nitrate, carbonate, bicarbonate, hydroxide, chloride, or bromide. The method 100 continues at step (c) with preparing a reaction mixture by mixing the resulting mixture in step (a) with the resulting mixture in step (b). In some embodiments, mixing in step (c) is carried out using at least one of mechanical, ultrasonic, vibration, ball-milling, and high-shear mixing methods. In some embodiments, preparing the reaction mixture in step (c) is carried out by mixing the first precursor, the second precursor, the first liquid, and the second liquid all together. The method 100 then proceeds to step (d) with introducing a third liquid to the reaction mixture in step (c), wherein the third liquid is selected from the group consisting of water, methanol, ethanol, propanol, butanol, formic acid, acetic acid, acetone, 2-butanone, acetonitrile, acrylonitrile, N-methyl-2-pyrrolidone, dimethylformamide, dimethyl sulfoxide, ethylene glycol, 2-methoxyethanol, 2-ethoxyethanol, 2-propoxyethanol, 2-butoxyethanol, propylene glycol, 1-methoxy-2-propanol, 1-ethoxy-2-propanol, methyl lactate, ethyl lactate, propylene carbonate, 4-butyrolactone, and combinations thereof. In other embodiments, the third liquid in step (d) is the same as the second liquid, which is the same as the first liquid, wherein the first liquid is selected from the group consisting of water, methanol, ethanol, propanol, butanol, formic acid, acetic acid, acetone, 2-butanone, acetonitrile, acrylonitrile, N-methyl-2-pyrrolidone, dimethylformamide, dimethyl sulfoxide, ethylene glycol, 2-methoxyethanol, 2-ethoxyethanol, 2-propoxyethanol, 2-butoxyethanol, propylene glycol, 1-methoxy-2-propanol, 1-ethoxy-2-propanol, methyl lactate, ethyl lactate, propylene carbonate, 4-butyrolactone, and combinations thereof. In some embodiments, introducing the third liquid in step (d) is carried out in batch processing. In other embodiments, introducing the third liquid in step (d) is carried out in continuous processing, wherein the resulting mixture in step (c) and the third liquid are continuously fed into a reactor; wherein the temperature of the reactor is kept at a temperature greater than or equal to 0° C. and less than or equal to 180° C.; and wherein continuously a product mixture comprising formamidinium iodide is removed from the reactor. In some embodiments, introducing the third liquid in step (d) is carried out in continuous processing, in which step (c) is omitted and wherein the mixture in step (a), the mixture in step (b), and the third liquid are continuously fed into a reactor; wherein the temperature of the reactor is kept at a temperature greater than or equal to 0° C. and less than or equal to 180° C.; and wherein continuously a product mixture comprising formamidinium iodide is removed from the reactor. The method 100 continues with step (e) with mixing the reaction mixture in step (d). In some embodiments, mixing in step (e) is carried out using at least one of mechanical, ultrasonic, vibration, pneumatic, ball-milling, emulsifying, high-shear mixing methods. In certain embodiments, mixing in step (e) is carried out with applying heat to the reaction mixture, wherein the temperature of the reaction mixture is adjusted to a temperature greater than or equal to 0° C. and less than or equal to 180° C. In some embodiments, mixing in step (e) is carried out with applying a pressure to the reaction mixture, wherein the pressure of the reaction mixture is adjusted to a value greater than or equal to 1 bar and less than or equal to 220 bar. The method 100 further continues with step (f) comprises collecting the solution which contains formamidinium iodide product. In some embodiments, collecting the solution containing formamidinium iodide product in step (f) may be achieved by any unit operation suitable for collecting a solution in either a liquid-solid mixture or a liquid-liquid mixture, with examples including decantation, centrifugation, separatory funnel, liquid-liquid extraction, filtration, and/or membrane separation. Finally, the example method 100 concludes at step (g) comprises isolating formamidinium iodide product by removing solvents in the product solution. In some embodiments, isolating formamidinium iodide product in step (g) may be achieved by distillation, evaporation, rotary evaporation, spray drying, freeze drying, and/or vacuum drying. In certain embodiments, the isolated formamidinium iodide in step (g) may be further purified using at least one of re-crystallization, sublimation, or chromatography.

According to some embodiments, after the isolating of formamidinium iodide product, the methods of making formamidinium iodide is continued with forming a perovskite film, wherein forming the perovskite film utilizes the isolated formamidinium iodide product as a precursor. According to some embodiments, the perovskite film is composed of a perovskite of formula: APbX3 wherein, A is a monovalent cation consisting of at least 70% formamidinium cation by mole fraction, Pb is lead(ii) cation, X is a monovalent halide anion consisting of at least 70% iodide anion by mole fraction. According to some embodiments, an optoelectronic and/or photoelectrochemical device is fabricated, wherein the optoelectronic and/or photoelectrochemical device comprises the perovskite film manufactured from the isolated formamidinium iodide precursor. In some embodiments, the optoelectronic device is a solar cell. In other embodiments, the device is a photoelectrochemical cell, which can be used for applications including, but not limited to, water splitting, lithium extraction, and/or carbon dioxide reduction.

While various embodiments have been presented in this disclosure, it should be recognized that the described systems and methods can be implemented in many other forms without deviating from the essence or scope of the disclosure. The provided examples are intended to be illustrative rather than limiting, and the intention is not to restrict the disclosure to the specific details provided. For instance, different elements or components may be merged or incorporated into another system, or certain features may be excluded or not implemented.

Additionally, the techniques, systems, subsystems, and methods described and depicted in various embodiments as distinct or separate may be combined or integrated with other systems, modules, techniques, or methods without altering the scope of the present disclosure. Items that are shown or discussed as being coupled, directly coupled, or communicating with each other may instead be indirectly coupled or communicate through an interface, device, or intermediate component, whether electrically, mechanically, or by other means. Other modifications, substitutions, and alterations can be identified by those skilled in the art and can be made without departing from the spirit and scope of the disclosure.

In addition, no restrictions are imposed on the construction or design details presented, except as defined in the claims below. It is clear that the illustrative embodiments may be altered or adapted, and all such variations fall within the scope and intent of the present invention. Specifically, any range of values (e.g., “from about a to about b,” or equivalently “from approximately a to b,” or “from approximately a-b”) disclosed herein is understood to encompass all subsets of that range, including every possible range within the broader one. Moreover, unless explicitly and clearly defined by the patentee, the terms used in the claims retain their plain, ordinary meaning.

1. A method for producing formamidinium iodide compound, comprising the steps of:
(a) mixing a first precursor, which contains formamidinium ions, with a first liquid;
(b) mixing a second precursor, which contains iodide ions, with a second liquid;
(c) preparing a reaction mixture by mixing the resulting mixture in step (a) with the resulting mixture in step (b);
(d) introducing a third liquid to the reaction mixture in step (c);
(e) mixing the reaction mixture in step (d);
(f) collecting the solution which contains formamidinium iodide product; and
(g) isolating formamidinium iodide product by removing solvents in the product solution.

2. The method of claim 1, wherein the counterions of formamidinium ions in the first precursor comprises at least one of formate, acetate, propanoate, benzoate, oxalate, lactate, citrate, sulfate, phosphate, nitrate, chloride, or bromide.

3. The method of claim 1, wherein the counterions of iodide ions in the second precursor comprises at least one of Li⁺, Na⁺, K⁺, Rb⁺, Cs⁺, H₃O⁺, NH₄⁺, CH₃NH₃⁺, (CH₃)₄N⁺, Ca²⁺, Mg²⁺, Fe²⁺, Mn²⁺, Zn²⁺, or Al³⁺.

4. The method of claim 1, wherein the first liquid is selected from the group consisting of water, methanol, ethanol, propanol, butanol, formic acid, acetic acid, acetone, 2-butanone, acetonitrile, acrylonitrile, N-methyl-2-pyrrolidone, dimethylformamide, dimethyl sulfoxide, and combinations thereof.

5. The method of claim 1, wherein the second liquid is the same as the first liquid.

6. The method of claim 1, wherein the second liquid is selected from the group consisting of water, methanol, ethanol, propanol, butanol, formic acid, acetic acid, acetone, 2-butanone, acetonitrile, acrylonitrile, N-methyl-2-pyrrolidone, dimethylformamide, dimethyl sulfoxide, and combinations thereof.

7. The method of claim 1, wherein the third liquid is selected from the group consisting of water, methanol, ethanol, propanol, butanol, formic acid, acetic acid, acetone, 2-butanone, acetonitrile, acrylonitrile, N-methyl-2-pyrrolidone, dimethylformamide, dimethyl sulfoxide, ethylene glycol, 2-methoxyethanol, 2-ethoxyethanol, 2-propoxyethanol, 2-butoxyethanol, propylene glycol, 1-methoxy-2-propanol, 1-ethoxy-2-propanol, methyl lactate, ethyl lactate, propylene carbonate, 4-butyrolactone, and combinations thereof.

8. The method of claim 1, wherein the third liquid is the same as the second liquid, which is the same as the first liquid, wherein the first liquid is selected from the group consisting of water, methanol, ethanol, propanol, butanol, formic acid, acetic acid, acetone, 2-butanone, acetonitrile, acrylonitrile, N-methyl-2-pyrrolidone, dimethylformamide, dimethyl sulfoxide, ethylene glycol, 2-methoxyethanol, 2-ethoxyethanol, 2-propoxyethanol, 2-butoxyethanol, propylene glycol, 1-methoxy-2-propanol, 1-ethoxy-2-propanol, methyl lactate, ethyl lactate, propylene carbonate, 4-butyrolactone, and combinations thereof.

9. The method of claim 1, wherein the first precursor contains cationic impurities comprising at least one of Li⁺, Na⁺, K⁺, Rb⁺, Cs⁺, H₃O⁺, NH₄⁺, CH₃NH₃⁺, (CH₃)₄N⁺, Be²⁺, Ca²⁺, Mg²⁺, Sr²⁺, Ba²⁺, Fe²⁺, Ni²⁺, Mn²⁺, Co²⁺, Zn²⁺, Al³⁺, or Bi³⁺.

10. The method of claim 1, wherein the second precursor contains anionic impurities comprising at least one of formate, acetate, propanoate, butanoate, pentanoate, hexanoate, benzoate, oxalate, malonate, lactate, citrate, hydrogen citrate, dihydrogen citrate, sulfate, hydrogen sulfate, phosphate, hydrogen phosphate, dihydrogen phosphate, nitrate, carbonate, bicarbonate, hydroxide, chloride, or bromide.

11. The method of claim 1, wherein the temperature of the reaction mixture is adjusted to a temperature greater than or equal to 0° C. and less than or equal to 180° C.

12. The method of claim 1, wherein step (a) and step (b) are omitted and the reaction mixture in step (c) is prepared by mixing the first precursor, the second precursor, the first liquid, and the second liquid all together.

13. The method of claim 1, wherein the third liquid is introduced in step (d) through a continuous process; wherein the mixture in step (c) and the third liquid are continuously fed into a reactor; wherein the temperature of the reactor is kept at a temperature greater than or equal to 0° C. and less than or equal to 180° C.; and wherein continuously a product mixture comprising formamidinium iodide is removed from the reactor.

14. The method of claim 1, wherein step (c) is omitted and the third liquid is introduced through a continuous process; wherein the mixture in step (a), the mixture in step (b), and the third liquid are continuously fed into a reactor; wherein the temperature of the reactor is kept at a temperature greater than or equal to 0° C. and less than or equal to 180° C.; and wherein continuously a product mixture comprising formamidinium iodide is removed from the reactor.

15. The method of claim 1, wherein a pressure is applied to the reaction mixture, wherein the pressure of the reaction mixture is adjusted to a value greater than or equal to 1 bar and less than or equal to 220 bar.

16. The method of claim 1, further comprising: after isolating the formamidinium iodide product, forming a perovskite film, wherein forming the perovskite film utilizes the isolated formamidinium iodide product as a precursor.

17. The method of claim 16, wherein the perovskite is a perovskite of formula: APbX3 wherein, A is a monovalent cation consisting of at least 70% formamidinium cation by mole fraction, Pb is lead(ii) cation, X is a monovalent halide anion consisting of at least 70% iodide anion by mole fraction.

18. An optoelectronic and/or photoelectrochemical device comprising a perovskite film manufactured using the method of claim 16.
